(19) United States
(12) Patent Application Publication
(10) Pub. No.: US 2025/0082955 A1
(43) Pub. Date: Mar. 13, 2025

(54) Photobiomodulation Light Therapy Device And System Thereof

(71) Applicant: Paul Salsberg, North York (CA)

(72) Inventor: Paul Salsberg, North York (CA)

(21) Appl. No.: 18/243,410

(22) Filed: Sep. 7, 2023

(51) Int. Cl. A61N 5/06

(52) U.S. Cl. CPC A61N 5/0616; A61N 2005/0626; A61N 2005/0648; A61N 2005/0651; A61N 2005/0663; A61N 2005/0666

(57) ABSTRACT

Disclosed herein is a light therapy system that includes a storage case and a photobiomodulation device. The photobiomodulation device includes a frame extending axially and longitudinally along a bridge present in middle of the frame. The photobiomodulation device includes a pair of reflectors attached to the frame, one of the pair of reflectors is adapted to encircle a first eye and other reflector in the pair of reflectors is adapted to encircle a second eye. The photobiomodulation device further includes light modules emitting light of specific wavelengths with a predefined irradiance level. The light modules may be placed at a predetermined distance on the pair of reflectors for emitting light penetrating into eyelids of the respective eye of the patient for treating the eyes and the surrounding skin of the patient for a pre-set time. The photobiomodulation device includes a button and a USB port connector attached to the frame.

### FIELD OF DISCLOSURE

The present disclosure relates generally to light therapy systems. More specifically, relates to a photobiomodulation light therapy device enhancing eye care practices and improving the ocular wellbeing of patients.

### BACKGROUND OF THE DISCLOSURE

Currently, photobiomodulation (PBM) technology is used in a small number of eye clinics, but it requires frequent in-office visits from patients. Moreover, PBM emerges as a game-changing technology in the field of universal eye care with the potential to transform eye care offerings, advance management of ocular conditions, and ultimately enhance people's lives.

By empowering self-care, individuals can actively participate in their eye health with the help of portable red light therapy devices. Individuals can enhance their regular optometric care by adding photobiomodulation to their daily routines, fostering general well-being and pro-active eye care.

The PBM technology used in the current invention can advance eye care procedures and raise patient ocular wellbeing. The goal of the current invention is to make affordable red light therapy systems or devices available in places with insufficient access to eye care resources through cooperation with eye care professionals. PBM can help users improve their overall health while also empowering them to supplement their regular eye care by incorporating it into their weekly routines.

There is a need for a light therapy system that solves the problems associated with the prior art.

### SUMMARY OF THE DISCLOSURE

The following is a summary description of illustrative embodiments of the invention. It is provided as a preface to assist those skilled in the art to more rapidly assimilate the detailed design discussion which ensues and is not intended in any way to limit the scope of the claims which are appended hereto in order to particularly point out the invention.

The present disclosure solves all the above major limitations of light therapy systems. According to illustrative embodiments, the present disclosure focuses on a light therapy system and a photobiomodulation light therapy device which overcomes the above-mentioned disadvantages or provide the users with a useful or commercial choice.

The objective of the present disclosure is an easy-to-use device and system thereof that allow patients to access the advantages of photobiomodulation (PBM) from the convenience of their homes and is readily distributable within a residence. In addition, people from all walks of life can prioritise their eye health regardless of their location or mobility as a result to the cost-effectiveness and portability of the device.

The objective of the current invention is to make affordable red light therapy equipment available in places with insufficient access to eye care. PBM helps users improve their overall health and gives patients the tools they need to supplement their regular optometric care by integrating it into weekly routines.

Another objective of the present invention is to provide a compact and user-friendly device enabling individuals to access the benefits of photobiomodulation from the comfort of their homes. The portability factor ensures that people from all walks of life, regardless of their location or mobility, can prioritize their eye health.

In light of the above, in one aspect of the present disclosure, a light therapy system is disclosed herein. The light therapy system includes a storage case. The light therapy system further includes a photobiomodulation device. The photobiomodulation device includes a frame extending axially and longitudinally along a bridge present in middle of the frame. The frame is configured to be supported on a nose of a patient when worn and positioned in front of a respective eye of the patient. The photobiomodulation device further includes a pair of reflectors attached to the frame, one of the pair of reflectors is adapted to encircle a first eye and other reflector in the pair of reflectors is adapted to encircle a second eye. The photobiomodulation device further includes light modules emitting light of specific wavelengths with a predefined irradiance level. The light modules may be placed at a predetermined distance on the pair of reflectors for emitting light penetrating into eyelids of the respective eye of the patient for treating the eyes and the surrounding skin of the patient for a pre-set time. The photobiomodulation device further includes a button coupled to the frame via an attachment. The button may be configured to turn ON and OFF the light modules. The photobiomodulation device further includes a USB port connector attached to the frame. The USB port connector may be configured to provide power input to the photobiomodulation device.

In one embodiment, the light therapy system further includes a head strap inserted into a side cavity present on both ends of the frame.

In one embodiment, the light therapy system further includes a supporting element associated with the pair of reflectors. Further, the supporting element is a downward V-shaped cushioned element complementary to the shape cut-out formed in the middle of the pair of reflectors.

In one embodiment, the light therapy system further includes a locking mechanism attached to the frame for coupling the pair of reflectors by inserting the edges pointing out of the pair of reflectors to the locking mechanism associated with the frame.

In one embodiment, the light therapy system further includes a storage case including a USB charging cord, a plug, the photobiomodulation device, and so forth.

In one embodiment, the light therapy system further includes a battery configured to provide power to the photobiomodulation device.

In one embodiment, the light therapy system further includes an indicator light present on the button configured to indicate power in the photobiomodulation device.

In one embodiment, the light modules comprise LED red lights emitting light of the specific wavelengths to stimulate cellular function within the eyes.

In one embodiment, the specific wavelength emits light in the red spectrum of about 670 nanometres (nm).

In one embodiment, the predefined irradiance level being 45 mw/cm².

In one embodiment, the pre-determined distance is an optimize distance from the light modules and the eye lid equal to about 3 to 4 millimetres.

In one embodiment, the pre-set time is about 3 minutes establishing a safe treatment time without the need of external timing devices.

In one embodiment, the head strap is a detachable and an adjustable head strap.

In light of the above, in another aspect of the present disclosure, a photobiomodulation light therapy device is disclosed herein. The photobiomodulation light therapy device includes a frame extending axially and longitudinally along a bridge present in middle of the frame. The frame is configured to be supported on a nose of a patient when worn and positioned in front of a respective eye of the patient. The photobiomodulation light therapy device further includes a pair of reflectors attached to the frame via a locking mechanism, one of the pair of reflectors is adapted to encircle a first eye and other reflector in the pair of reflectors is adapted to encircle a second eye. The photobiomodulation light therapy device further includes a supporting element associated with the pair of reflectors, the supporting element is a downward V-shaped cushioned element complementary to the shape cut-out formed in the middle of the pair of reflectors. The photobiomodulation light therapy device further includes a locking mechanism attached to the frame for coupling the pair of reflectors by inserting the edges pointing out of the pair of reflectors to the locking mechanism associated with the frame. The photobiomodulation light therapy device further includes light modules having LED red lights emitting light of specific wavelengths with a predefined irradiance level to stimulate cellular function within the eyes placed at a predetermined distance on the pair of reflectors for emitting light penetrating into eyelids of the respective eye of the patient for treating the eyes and the surrounding ocular region including skin of the patient for a pre-set time. The photobiomodulation light therapy device further includes a button coupled to the frame and connected to the button configured to turn ON and OFF the light modules. The photobiomodulation light therapy device further includes a button coupled to the frame via an attachment. The button may be configured to turn ON and OFF the light modules. The photobiomodulation light therapy device further includes a USB port connector attached to the frame. The USB port connector may be configured to provide power input to the photobiomodulation light therapy device. The photobiomodulation light therapy device further includes an adjustable head strap inserted into a side cavity present on both ends of the frame.

In one embodiment, the photobiomodulation light therapy device further includes a battery configured to provide power to the photobiomodulation device.

In one embodiment, the photobiomodulation light therapy device further includes an indicator light present on the button configured to indicate power of the photobiomodulation light therapy device.

In one embodiment, the specific wavelength emits light in the red spectrum of about 670 nanometres (nm).

In one embodiment, the head strap is a detachable head strap.

In one embodiment, the pre-set time is about 3 minutes establishing a safe treatment time without the need of external timing devices.

In one embodiment, the photobiomodulation light therapy device automatically turns off when the pre-set time reaches 3 minutes.

In one embodiment, the predefined irradiance level being 45 mw/cm².

In one embodiment, the frame is made from an ultra-lightweight, flexible, resistant to breakage thermoplastic material configured to adapt any face shape of the patient for maximum comfort.

In one embodiment, the pre-determined distance is an optimize distance between the light modules and the eye lid that is equal to about 3 to 4 millimetres.

The present invention provides an accessible red light therapy device to areas lacking adequate eye care resources. By breaking down barriers and improving accessibility, the light therapy system empowers individuals to take control of their eye health.

These and other advantages will be apparent from the present application of the embodiments described herein.

The preceding is a simplified summary to provide an understanding of some embodiments of the present invention. This summary is neither an extensive nor exhaustive overview of the present invention and its various embodiments. The summary presents selected concepts of the embodiments of the present invention in a simplified form as an introduction to the more detailed description presented below. As will be appreciated, other embodiments of the present invention are possible utilizing, alone or in combination, one or more of the features set forth above or described in detail below.

These elements, together with the other aspects of the present disclosure and various features are pointed out with particularity in the claims annexed hereto and form a part of the present disclosure. For a better understanding of the present disclosure, its operating advantages, and the specified object attained by its uses, reference should be made to the accompanying drawings and descriptive matter in which there are illustrated exemplary embodiments of the present disclosure.

### BRIEF DESCRIPTION OF THE DRAWINGS

To describe the technical solutions in the embodiments of the present disclosure or in the prior art more clearly, the following briefly describes the accompanying drawings required for describing the embodiments or the prior art. Apparently, the accompanying drawings in the following description merely show some embodiments of the present disclosure, and a person of ordinary skill in the art can derive other implementations from these accompanying drawings without creative efforts. All of the embodiments or the implementations shall fall within the protection scope of the present disclosure.

The advantages and features of the present disclosure will become better understood with reference to the following detailed description taken in conjunction with the accompanying drawing, in which:

FIG. 1 illustrates a perspective view of a light therapy system, in accordance with an exemplary embodiment of the present disclosure;

FIG. 2A illustrates a perspective view of a photobiomodulation device, in accordance with an exemplary embodiment of the present disclosure;

FIG. 2B illustrates a back view of the photobiomodulation device, in accordance with an exemplary embodiment of the present disclosure;

FIG. 2C illustrates a bottom view of a photobiomodulation device, in accordance with an exemplary embodiment of the present disclosure;

FIG. 3A illustrates a perspective view of a pair of reflectors, in accordance with an exemplary embodiment of the present disclosure;

FIG. 3B illustrates a cross sectional via of light modules placed strategically in the pair of reflectors, in accordance with an exemplary embodiment of the present disclosure;

FIG. 3C illustrates a bottom view of a supporting element attached to the pair of reflectors, in accordance with an exemplary embodiment of the present disclosure;

FIG. 3D illustrates a side view of the button coupled to the frame via an attachment, in accordance with an exemplary embodiment of the present disclosure; and

FIG. 3E illustrates a front view of a detachable and adjustable head strap, in accordance with an exemplary embodiment of the present disclosure.

Like reference, numerals refer to like parts throughout the description of several views of the drawing.

The light therapy system and a photobiomodulation light therapy device is illustrated in the accompanying drawings, which like reference letters indicate corresponding parts in the various figures. It should be noted that the accompanying figure is intended to present illustrations of exemplary embodiments of the present disclosure. This figure is not intended to limit the scope of the present disclosure. It should also be noted that the accompanying figure is not necessarily drawn to scale.

### DETAILED DESCRIPTION OF THE DISCLOSURE

The following is a detailed description of embodiments of the disclosure depicted in the accompanying drawings. The embodiments are in such detail as to communicate the disclosure. However, the amount of detail offered is not intended to limit the anticipated variations of embodiments; on the contrary, the intention is to cover all modifications, equivalents, and alternatives falling within the spirit and scope of the present disclosure.

In the following description, numerous specific details are set forth in order to provide a thorough understanding of the embodiments of the present disclosure. It may be apparent to one skilled in the art that embodiments of the present disclosure may be practiced without some of these specific details.

Various terms as used herein are shown below. To the extent a term is used, it should be given the broadest definition persons in the pertinent art have given that term as reflected in printed publications and issued patents at the time of filing.

The terms “a” and “an” herein do not denote a limitation of quantity, but rather denote the presence of at least one of the referenced items.

The terms “having”, “comprising”, “including”, and variations thereof signify the presence of a component.

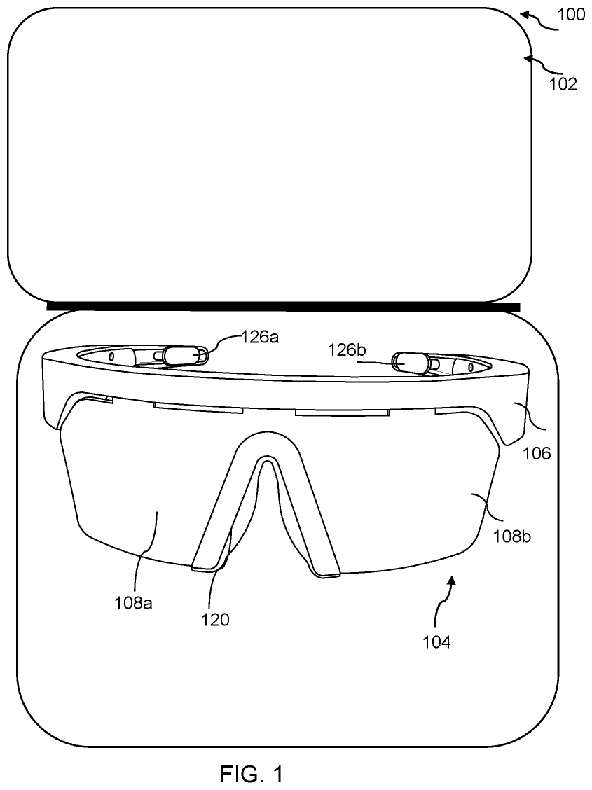

Referring now to FIG. 1 to FIG. 3E to describe various exemplary embodiments of the present disclosure. FIG. 1 illustrates a perspective view of a light therapy system 100, in accordance with an exemplary embodiment of the present disclosure. The light therapy system 100 comprises a storage case 102, a photobiomodulation device 104 (hereinafter also referred to as a photobiomodulation light therapy device 104), a frame 106, a pair of reflectors 108a, 108b, light modules 110, a button 112, an attachment 114, a USB port connector 116, a head strap 118, a supporting element 120, and a locking mechanism 122.

The storage case 102 may be configured to store a plurality of components associated with the light therapy system 100. According to embodiments of the present invention, the storage case 102 may include the plurality of components such as but not limited to a USB charging cord, a plug, the photobiomodulation device 104, and so forth.

The light therapy system 100 further includes the photobiomodulation device 104 which may be a rechargeable and cordless device. The photobiomodulation represents a cutting-edge approach to eye care, merging technology with optometry. As advancements continue, the integration of red-light therapy into routine eye examinations and treatment protocols holds early detection and management of various eye conditions.

The photobiomodulation device 104 may be a portable red light therapy device striving to make eye care accessible and inclusive for all individuals, regardless of their location or circumstances. By leveraging the benefits of photobiomodulation on the posterior and anterior segments of the eyes, the optimal eye health is within everyone's reach.

The photobiomodulation device 104 includes the frame 106 extending axially and longitudinally along a bridge present in middle of the frame 106. The frame 106 may be configured to be supported on a nose of a patient when worn and positioned in front of a respective eye of the patient. In one embodiment, the frame 106 may be made of a single piece material. In other embodiment, the frame 106 may be made of one or more parts and screwed together via screws 128 to form a complete frame 106.

According to embodiments of the present invention, the frame 106 may be made from an ultra-lightweight, flexible, resistant to breakage thermoplastic material configured to adapt any face shape of the patient for maximum comfort.

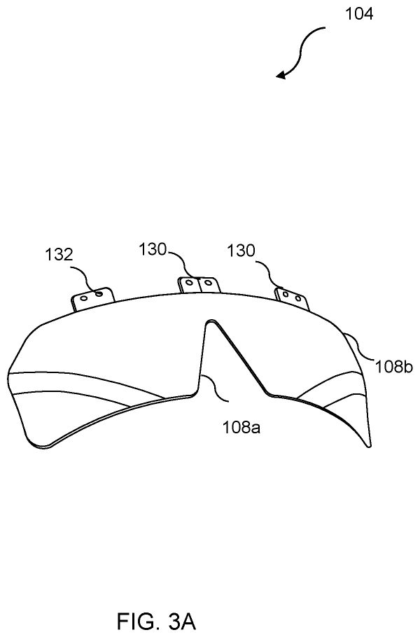

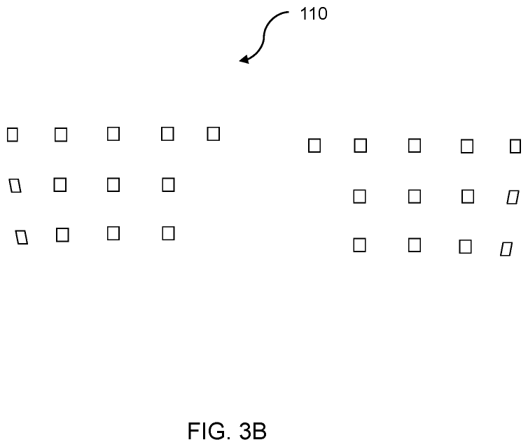

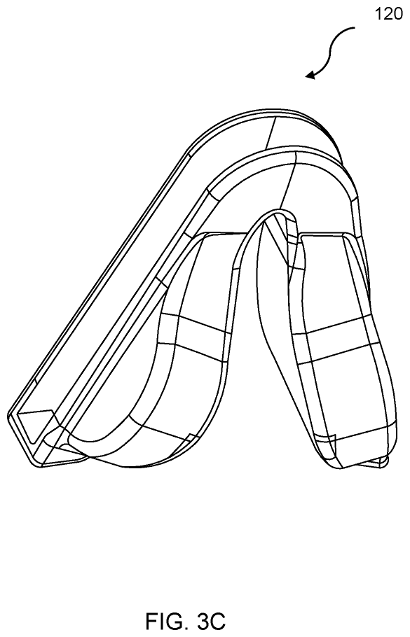

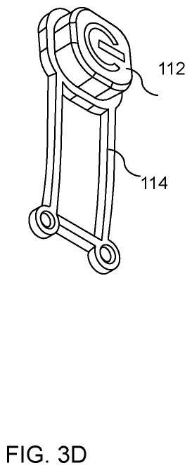

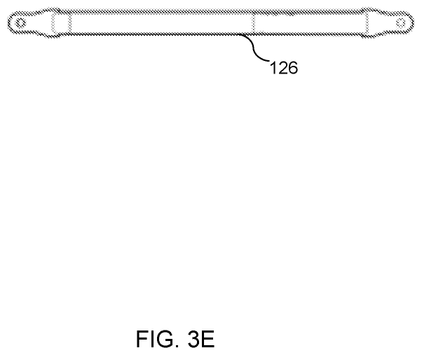

The head strap 126 may be inserted into a side cavity 124 present on both ends 106a, 106b of the frame 106 and remain attached via a sliding element 126a, 126b present on both ends 106a, 106b of the frame 106, as seen in FIG. 3E. The sliding element 126a, 126b attach and detach the head strap 126 from the frame 106. In an exemplary embodiment, the head strap 126 may be a detachable and an adjustable head strap.

The pair of reflectors 108a, 108b may be attached to the frame 106. One of the pair of reflectors 108a may be adapted to encircle a first eye and other reflector 108b in the pair of reflectors 108a, 108b is adapted to encircle a second eye.

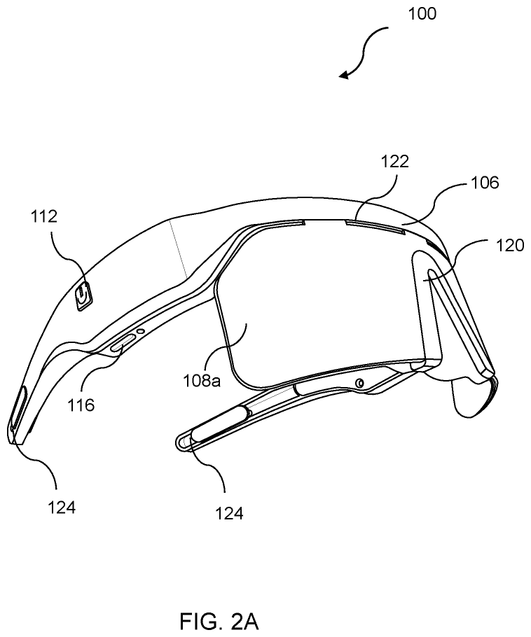

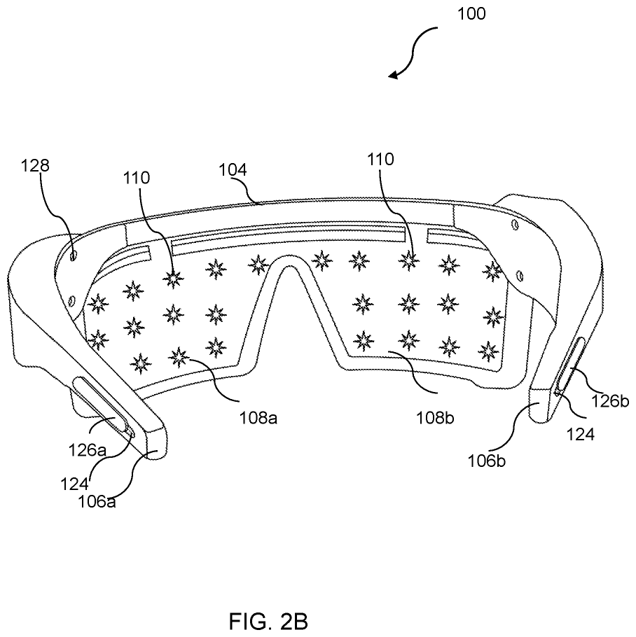

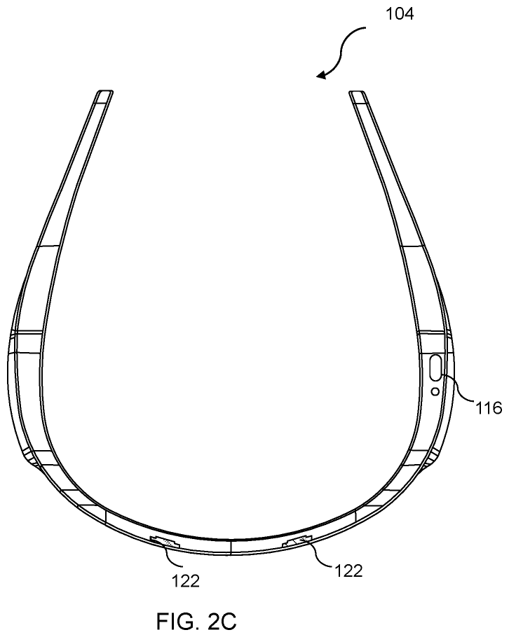

Further referring to FIG. 2A, the supporting element 120 may be associated with the pair of reflectors 120. In one embodiment, the supporting element 120 may be a downward V-shaped cushioned element complementary to the shape cut-out formed in the middle of the pair of reflectors 108a, 108b, as seen FIG. 3C.

According to an embodiment, the locking mechanism 122 may be attached to the frame 106 for coupling the pair of reflectors 108a, 108b by inserting the edges 130 pointing out of the pair of reflectors 108a, 108b to the locking mechanism 122 associated with the frame 106, as seen in FIG. 2C and FIG. 3A. Additionally, the edge may comprise holes 132 in which a locking element present in the locking mechanism may be inserted so as to attach the pair of reflectors 108a, 108b to the frame 106.

The light modules 110 emitting light of specific wavelengths with a predefined irradiance level. In one embodiment, the predefined irradiance level being 45 mw/cm².

The irradiance level or power density is designed for eye use and thus not exceeding safe exposure limits.

The light modules 110 may be placed at a predetermined distance on the pair of reflectors 108a, 108b for emitting light penetrating into eyelids of the respective eye of the patient for treating the eyes and the surrounding skin of the patient for a pre-set time. The emitted light improves blood flow to all surrounding skin areas including the glabellar lines (between your eye brows), under the eyes and the lateral canthal lines (known as crow's feet).

In one embodiment, the pair of reflectors 108a, 108b may comprise 26 strategically placed light modules 110 that may be LED red lights emitting light at 670 nm, as seen in FIG. 2B and FIG. 3B. The light modules 110 may be single-core LED lights with an operating temperature of 0° C. to 40° C. or 32° F. to 104° F.

The light modules 110 may comprise medical-grade light emitting diodes (LEDs) delivering superior irradiance at optimal wavelengths while staying cool to the touch so the patient may wear the photobiomodulation device 104 daily in total comfort.

In one exemplary embodiment, the storage and transport temperature may be −5° C. to 40° C. or 23° F. to 104° F. and storage and transport humidity may be 20% to 85% RH without condensation. Further the operating humidity may be 30% to 95% RH without condensation.

The pre-determined distance may be an optimize distance from the light modules 110 and the eye lid equal to about 3 to 4 millimetres. The pre-set time may be about 3 minutes establishing a safe treatment time without the need of external timing devices.

The photobiomodulation device 104 may be automatically turned off when the pre-set time reaches 3 minutes.

The light modules 110 may comprise LED red lights emitting light of the specific wavelengths to stimulate cellular function within the eyes. In one embodiment, the specific wavelength emits light in the red spectrum of about 670 nanometres (nm).

Referring to FIG. 3D, the photobiomodulation device 104 further includes the button 112 coupled to the frame 106 via an attachment 114. The button 112 may be configured to turn ON and OFF the light modules 110.

The USB port connector 116 attached to the frame 106. The USB port connector 116 may be configured to provide power input to the photobiomodulation device 104. The USB port connector 116 may be C type USB port providing power input of 5V or 2A via a charging cable with a battery charging time of 60 minutes.

The photobiomodulation device 104 further includes the battery that may be configured to provide power to the photobiomodulation device 104.

The photobiomodulation device 104 further includes an indicator light present on the frame configured to indicate power in the photobiomodulation device 104.

In an exemplary embodiment, the indication light may be present in the button 112 attached to the frame 106. In one embodiment, the photobiomodulation device 104 may have a weight of 3.5 oz.

In one of the best modes of operation, the present invention utilizes an optimal dose of red-light exposure (taking into account the wavelength of light used, distance from the light modules 110 to the eyes, the intensity of light, duration of each treatment, and frequency of treatment) for producing the most favourable biologic outcome.

The goal of the current invention is an easy-to-use, non-invasive, 100 percent UV free, FDA-approved system that allows patients to access the advantages of photobiomodulation (PBM) from the convenience of their homes and is readily distributable within a residence.

Additionally, the portability and cost-effectiveness make it possible for people from all walks of life to prioritise their eye health regardless of their location or mobility. This non-invasive approach harnesses the power of light to promote healing, reduce inflammation, enhance blood flow, and support overall eye health. By providing targeted PBM to the posterior and anterior segments of the eyes, we can address a range of ocular conditions and promote general well-being.

Individuals can empower self-care and actively participate in their eye health by using portable red light therapy devices. Individuals can supplement their routine optometric care with photobiomodulation, which promotes overall wellness and preventative eye care. The PBM technology used in the current invention can improve patient ocular health and eye care procedures.

The light therapy system 100 allows wireless treatments to be delivered at home or travel. The light therapy system 100 has a compact and portable design that enhances convenience and usability. The light therapy system 100 is super easy to use, just put on the photobiomodulation device 104, press the button 112 and relax, the patient doesn't have to keep his/her eyes open and the light emitted by the light modules will penetrate the eyelid.

The photobiomodulation device 104 is a single-wavelength light emitting diode (LED) instrument intended for use by patients for photobiomodulation (PBM) treatment of the eye. The photobiomodulation device 104 may deliver a predetermined PBM treatment to the eye and retinal tissue of the subject through the closed eyelid. Light therapies are currently utilised extensively in both human and animal medicine. Photobiomodulation (PBM) has utilised monochromatic (laser) and narrow band (LED) light in the optical spectral region of 600-900 nm to treat a variety of biological targets in a non-destructive and nonthermal manner.

The light therapy system 100 is designed to emit light with a wavelength of 670 nm. The selection of a 670 nm wavelength is due to its interaction with Cytochrome C oxidase (CCO). CCO is an enzyme of the inner mitochondrial membrane, a component of Complex IV of the electron transport chain, and a target chromophore for both of these frequencies. Red light with a wavelength of 670 nanometres is known to be absorbed by copper CuB within CCO and has been shown to increase O2 binding at the active site. Both wavelengths independently stimulate CCO enzyme activity, restoring mitochondrial membrane potential and resulting in increased ATP production.

While the invention has been described in connection with what is presently considered to be the most practical and various embodiments, it will be understood that the invention is not to be limited to the disclosed embodiments, but on the contrary, is intended to cover various modifications and equivalent arrangements included within the scope of the appended claims.

A person of ordinary skill in the art may be aware that, in combination with the examples described in the embodiments disclosed in this specification, units and algorithm steps may be implemented by electronic hardware, computer software, or a combination thereof.

The foregoing descriptions of specific embodiments of the present disclosure have been presented for purposes of illustration and description. They are not intended to be exhaustive or to limit the present disclosure to the precise forms disclosed, and many modifications and variations are possible in light of the above teaching. The embodiments were chosen and described to best explain the principles of the present disclosure and its practical application, and to thereby enable others skilled in the art to best utilize the present disclosure and various embodiments with various modifications as are suited to the particular use contemplated. It is understood that various omissions and substitutions of equivalents are contemplated as circumstances may suggest or render expedient, but such omissions and substitutions are intended to cover the application or implementation without departing from the scope of the present disclosure.

In a case that no conflict occurs, the embodiments in the present disclosure and the features in the embodiments may be mutually combined. The foregoing descriptions are merely specific implementations of the present disclosure, but are not intended to limit the protection scope of the present disclosure. Any variation or replacement readily figured out by a person skilled in the art within the technical scope disclosed in the present disclosure shall fall within the protection scope of the present disclosure. Therefore, the protection scope of the present disclosure shall be subject to the protection scope of the claims.

What is claimed is:

1. A photobiomodulation light therapy device comprising:
a frame extending axially and longitudinally along a bridge present in middle of the frame, the frame is configured to be supported on a nose of a patient when worn and positioned in front of a respective eye of the patient;
a pair of reflectors attached to the frame via a locking mechanism, one of the pair of reflectors is adapted to encircle a first eye and other reflector in the pair of reflectors is adapted to encircle a second eye;
a supporting element associated with the pair of reflectors, the supporting element is a downward V-shaped cushioned element complementary to the shape cut-out formed in the middle of the pair of reflectors;
a locking mechanism attached to the frame for coupling the pair of reflectors by inserting the edges pointing out of the pair of reflectors to the locking mechanism associated with the frame;
light modules having LED red lights emitting light of specific wavelengths with a predefined irradiance level to stimulate cellular function within the eyes placed at a predetermined distance on the pair of reflectors for emitting light penetrating into eyelids of the respective eye of the patient for treating the eyes and the surrounding ocular region including skin of the patient for a pre-set time;
a button coupled to the frame via an attachment, the button is configured to turn ON and OFF the light modules;
a USB port connector attached to the frame, the USB port connector configured to provide power input to the photobiomodulation light therapy device; and
an adjustable head strap inserted into a side cavity present on both ends of the frame.

2. The photobiomodulation light therapy device of claim 1, further comprising: a battery configured to provide power to the photobiomodulation device; and an indicator light present on the button configured to indicate power of the photobiomodulation light therapy device.

3. The photobiomodulation light therapy device of claim 1, wherein the specific wavelength emits light in the red spectrum of about 670 nanometres (nm).

4. The photobiomodulation light therapy device of claim 1, wherein the head strap is a detachable head strap.

5. The photobiomodulation light therapy device of claim 1, wherein the pre-set time is about 3 minutes establishing a safe treatment time without the need of external timing devices.

6. The photobiomodulation light therapy device of claim 1, wherein the photobiomodulation light therapy device automatically turns off when the pre-set time reaches 3 minutes.

7. The photobiomodulation light therapy device of claim 1, wherein the predefined irradiance level being 45 mw/cm².

8. The photobiomodulation light therapy device of claim 1, wherein the frame is made from an ultra-lightweight, flexible, resistant to breakage thermoplastic material configured to adapt any face shape of the patient for maximum comfort.

9. The photobiomodulation light therapy device of claim 1, wherein the pre-determined distance is an optimize distance between the light modules and the eye lid that is equal to about 3 to 4 millimetres.

10. A light therapy system comprising:
a storage case; and
a photobiomodulation device including:
a frame extending axially and longitudinally along a bridge present in middle of the frame, the frame is configured to be supported on a nose of a patient when worn and positioned in front of a respective eye of the patient;
a pair of reflectors attached to the frame, one of the pair of reflectors is adapted to encircle a first eye and other reflector in the pair of reflectors is adapted to encircle a second eye;
light modules emitting light of specific wavelengths with a predefined irradiance level, the light modules placed at a predetermined distance on the pair of reflectors for emitting light penetrating into eyelids of the respective eye of the patient for treating the eyes and the surrounding skin of the patient for a pre-set time;
a button coupled to the frame via an attachment, the button is configured to turn ON and OFF the light modules; and
a USB port connector attached to the frame, wherein the USB port connector configured to provide power input to the photobiomodulation device.

11. The light therapy system of claim 10, further comprising:
a head strap inserted into a side cavity present on both ends of the frame;
a supporting element associated with the pair of reflectors, the supporting element is a downward V-shaped cushioned element complementary to the shape cut-out formed in the middle of the pair of reflectors; and
a locking mechanism attached to the frame for coupling the pair of reflectors by inserting the edges pointing out of the pair of reflectors to the locking mechanism associated with the frame.

12. The light therapy system of claim 10, further comprising a storage case including a USB charging cord, a plug, the photobiomodulation device, or any combination thereof.

13. The light therapy system of claim 10, further comprising a battery configured to provide power to the photobiomodulation device.

14. The light therapy system of claim 10, further comprising an indicator light present on the button configured to indicate power in the photobiomodulation device.

15. The light therapy system of claim 10, wherein the light modules comprise LED red lights emitting light of the specific wavelengths to stimulate cellular function within the eyes.

16. The light therapy system of claim 10, wherein the specific wavelength emits light in the red spectrum of about 670 nanometres (nm).

17. The light therapy system of claim 10, wherein the predefined irradiance level being 45 mw/cm².

18. The light therapy system of claim 10, wherein the pre-determined distance is an optimize distance from the light modules and the eye lid equal to about 3 to 4 millimetres.

19. The light therapy system of claim 10, wherein the pre-set time is about 3 minutes establishing a safe treatment time without the need of external timing devices.

20. The light therapy system of claim 10, wherein the head strap is a detachable and an adjustable head strap.
